(19) United States
(12) Patent Application Publication
(10) Pub. No.: US 2024/0023863 A1
(43) Pub. Date: Jan. 25, 2024

(54) BIOLOGICAL MONITORING ELECTRODE AND WEARABLE DEVICE

(71) Applicant: GOERTEK INC., Weifang, Shandong (CN)

(72) Inventors: Lianheng HAN, Weifang, Shandong (CN); Jizhi SONG, Weifang, Shandong (CN); Anlu ZHAO, Weifang, Shandong (CN); Qiang REN, Weifang, Shandong (CN); Xiaogang WU, Weifang, Shandong (CN)

(21) Appl. No.: 18/256,444

(22) PCT Filed: Aug. 25, 2021
(86) PCT No.: PCT/CN2021/114455

§ 371 Date: Jun. 8, 2023

(30) Foreign Application Priority Data
Dec. 8, 2020 (CN) 202011422296.8

(51) Int. Cl. A61B 5/263; H05K 9/00; A61B 5/28; A61B 5/00; A61B 5/26; A61B 5/332

(52) U.S. Cl. CPC A61B 5/263; H05K 9/0086; A61B 5/28; A61B 5/681; A61B 5/26; A61B 5/332

(57) ABSTRACT

A biological monitoring electrode including an electrode body, wherein a surface of the electrode body is coated with an insulating anti-interference material layer, the insulating anti-interference material layer has a first notch, and the electrode body is exposed by the first notch to be contacted with a user's skin to acquire biological information. For example, a user's finger contacts the electrode body through the first notch as a signal input. During the biological signal acquisition process, since the surface of the electrode body is coated with the insulating anti-interference material layer, and the insulating anti-interference material layer may achieve the function of insulating and shielding electromagnetic interference environment interference, therefore the biological monitoring electrode may effectively shield external noise interference, and reduce the influence of external noise on acquisition of biological information, such as electrocardiogram signals, thereby improving the accuracy of measurement results.

The present disclosure claims the priority of the Chinese Patent Application No. 202011422296.8, titled “biological monitoring electrode and wearable device” filed to China National Intellectual Property Administration on Dec. 8, 2020, the entire contents of which are incorporated herein by reference.

### TECHNICAL FIELD

The present disclosure relates to a technical field of biological monitoring, more specifically, relates to a biological monitoring electrode, and also relates to a wearable device.

### DESCRIPTION OF RELATED ART

At present, there are more and more wearable devices such as watch with biological monitoring function. Taking a watch as an example, one electrode is disposed on the lower case of the watch, and another electrode is disposed on the edge of the upper case of the watch, a button, or a watch strap. The device obtains electro cardiac information (ECG information) by measuring the potential difference between the user's wrist and the fingers of the other hand. The electrode of watches often uses dry electrodes, usually metal or conductive ceramic. Since dry electrodes are sensitive to displacement, metal and ceramic having different materials and areas may affect the test results. Therefore, it is necessary to design reasonable electrodes to avoid interference signal, otherwise it will have a significant influence on the measurement results.

Some existing watches with biological monitoring functions usually use three electrodes to improve signal definition and reduce noise, with two electrodes connected to the left and right hands respectively, and the third electrode is connected to other body portions except the left hand and right hands to eliminate power-line interference. However, the connection of the third electrode increases the difficulty of the watch structure design. For dual electrodes structure, some of them also use electrodes made of pure silver, silver alloy, or silver plated materials, and the electrocardiogram signals acquired by them have a quality equivalent to the test results of three electrodes. However, the three electrode system and silver material dual electrodes may still cause certain noise in the acquired electrocardiogram signals due to the influence of external noise on the electrocardiogram signals.

In summary, the problem that needs to be solved at present is how to effectively solve the problem such as noise affects measurement results when electrodes of wearable device collect biological information.

### SUMMARY

In view of the above, a first purpose of the present disclosure is to provide a biological monitoring electrode, which may effectively solve the problem that noise affects the measurement results when the electrode of a wearable device collecting biological information. The second purpose of the present disclosure is to provide a wearable device including the biological monitoring electrode as described above.

In order to achieve the first purpose described above, the present disclosure provides the following technical solution.

A biological monitoring electrode includes an electrode body, a surface of the electrode body is coated with an insulating anti-interference material layer, the insulating anti-interference material layer has a first notch, and the electrode body is exposed by the first notch to be contacted with a user's skin to acquire biological information.

Preferably, in the biological monitoring electrode described above, the electrode body has a convex portion protruding from the first notch.

Preferably, in the biological monitoring electrode described above, the insulating anti-interference material layer includes an insulating coating layer as a top layer and a metal mesh layer and/or a metal foil layer disposed between the insulating coating layer and the electrode body.

Preferably, in the biological monitoring electrode described above, the metal foil layer is fixed to a surface of the electrode body, and the metal mesh layer is fixed to a surface of the metal foil layer.

Preferably, in the biological monitoring electrode described above, the metal mesh layer is a copper mesh layer, and the metal foil layer is a copper foil layer.

Preferably, in the biological monitoring electrode described above, the insulating coating layer is a plastic layer.

Preferably, in the biological monitoring electrode described above, it further includes an adhesive layer for bonding and fixing the electrode body to the metal foil layer or the metal mesh layer.

Preferably, in the biological monitoring electrode described above, the adhesive layer is a plastic layer formed by spray molding.

Preferably, in the biological monitoring electrode described above, the electrode body serves as a button for a wearable device, and the insulating anti-interference material layer has a second notch at a position corresponding to a bottom end of the electrode body, the electrode body is exposed by the second notch to be contacted and electrically connect with a tactile switch of the wearable device.

The biological monitoring electrode provided by the present disclosure includes an electrode body and an insulating anti-interference material layer. Wherein the insulating anti-interference material layer wraps the surface of the electrode body, and the insulating anti-interference material layer has a first notch, the electrode body is exposed by the first notch to be contacted with the user's skin so as to acquire biological information.

In application of the biological monitoring electrode provided by the present disclosure, the insulating interference layer has the first notch, and the electrode body is exposed by the first notch, therefore, users can acquire biological signals by contacting the electrode body, for example, the user's finger contacts the electrode body through the first notch, as a signal input. In the process during biological signal acquisition, since the surface of the electrode body is coated with the insulating anti-interference material layer, which may function to insulate and shield environmental interference, therefore, the biological monitoring electrode may effectively shield external noise interference, reducing the impact of external noise on the acquisition of biological information, such as electrocardiogram signals, to improve the accuracy of measurement results.

In order to achieve the second purpose described above, the present disclosure also provides a wearable device that includes the biological monitoring electrode according to any one of items describe above. Since the biological monitoring electrode described above has the above technical effects, the wearable device having the biological monitoring electrode also have the corresponding technical effects.

### BRIEF DESCRIPTION OF DRAWINGS

In order to more clearly illustrate the embodiments of the present disclosure or the technical solutions in the art, the drawings required to be used for the content of the embodiments or the prior art will be briefly introduced in the following. Obviously, the drawings in the following description are merely a part of the drawings of the present disclosure and for those of ordinary skill in the art, other drawings may also be obtained from the provided drawings without any creative effort.

FIG. 1 is a schematic diagram illustrating an installed state of a biological monitoring electrode according to a specific embodiment of the present disclosure.

FIG. 2 is a structural schematic diagram of Section A-A in FIG. 1.

The reference signs in the drawings: device housing 1; button 2; button support 3; sealing ring 4; protruding portion 5; tactile switch 6; circuit board 7; substrate 8; insulating anti-interference material layer 9; electrode body 100; metal foil layer 200; metal mesh layer 300; insulating coating layer 400; and adhesive layer 500.

### DETAILED DESCRIPTIONS

The embodiment of the present disclosure discloses a biological monitoring electrode which may reduce the influence of external interference in acquired biological signals, such as electrocardiogram signals.

Technical solutions of embodiments of the present disclosure will be described below in combination with the drawings in the embodiments of the present disclosure. Obviously, the described embodiments are only a part of the embodiments of the present disclosure, rather than all the embodiments. Based on the embodiments in the present disclosure, all other embodiments obtained by those of ordinary skill in the art without creative efforts shall fall within the protection scope of the present disclosure.

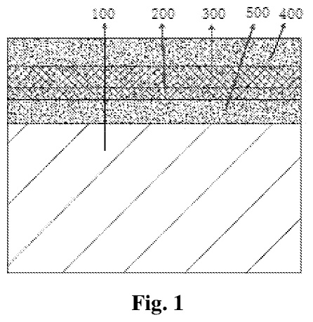

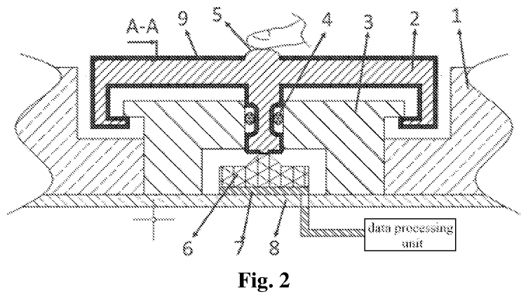

Referring to FIGS. 1 and 2, FIG. 1 is a schematic diagram illustrating an installed state of the biological monitoring electrode according to a specific embodiment of the present disclosure, and FIG. 2 is a schematic diagram of Section A-A in FIG. 1.

In a specific embodiment, the biological monitoring electrode provided by the present disclosure includes an electrode body 100 and an insulating anti-interference material layer 9.

Here, the electrode body 100, i.e., the main component of the biological monitoring electrode contacts the user's skin to acquire the user's biological information, in particular, electrocardiogram information, etc. The acquisition of biological information herein includes both the direct acquisition of biological information and the indirect reflection of biological information through the acquisition of signals such as voltages. The operation principle of the electrode body 100 is known to those skilled in the art, and thus will not be described in detail herein. The electrode body 100 may be mounted on wearable devices such as watches. Specifically, the electrode body 100 may be used alone as a biological signal, or the electrode may also used as a button 2 for wearable device in the meanwhile to save structural design and achieve corresponding triggering functions by pressing or lifting the button 2 while being used for biological information collection. The specific structure and operation principle of the button 2 may also similar to those in the art.

The insulating anti-interference material layer 9 warps the surface of the electrode body 100. It should be noted that the surface herein not only includes the exposed upper surface of the electrode body 100 in an installed state, but also includes the surface of the electrode body 100 opposite to the device housing 1. That is, the insulating anti-interference material layer 9 is provided on the surface of the entire electrode body 100 in all directions except the contacting areas, to ensure the electrode body is insulated from the device housing 1, so as to avoid the risk of electrical conduction between the electrode body 100 and the housing 1 made of metal, and further improve the operation safety of the device. The insulating anti-interference material layer 9 is used for insulating and shielding environmental interference signals. Specifically, the insulating anti-interference material layer 9 is used to shield external electromagnetic interference and radio frequency interference to ensure the precision and effectiveness of biological signals, such as electrocardiogram.

In order to ensure the function of the electrode body 100 in acquiring biological signals, a first notch is formed in the insulating anti-interference material layer 9, and the electrode body 100 is exposed by the first notch to be contacted with the user's skin to acquire biological information. The specific size and shape of the first notch may be set according to the area that the user's skin contacts the electrode body 100. For example, when the user's finger contacts the electrode body 100, the size and shape of the first notch may be set corresponding to the size of the finger pulp. It is also possible to perform other settings according to the size of the first notch to meet the requirements of biological signal acquisition. Of course, in this case, the smaller the size of the first notch, the better the electromagnetic shielding effect of the corresponding insulating anti-interference material layer 9.

In the application of the biological monitoring electrode provided by the present disclosure, the insulating interference layer has a first notch, and the electrode body 100 is exposed by the first notch, and thus users may acquire biological signals by contacting the electrode body 100 (e.g., the user's finger contacts the electrode body 100 through the first notch) as a signal input. In the process during biological signal acquisition, since the surface of the electrode body 100 is coated with the insulating anti-interference material layer 9, which may service to insulate and shield environmental interference, so that the biological monitoring electrode may effectively shield external noise interference, reducing the impact of external noise on the acquisition of biological information, such as electrocardiogram signals, to improve the accuracy of measurement results.

In order to be easily contacted with the user's skin, specifically, the electrode body 100 has a convex portion 5 that protrudes from the first notch. The shape of the protruding portion 5 may be formed according to the actual situation, for example, the protruding portion 5 may be formed to have an arc-shaped top surface. The protruding portion 5 protrudes from the first notch, so that the user may easily contact the electrode body 100 to perform signal acquisition. Specifically, the convex portion 5 may be positioned at the upper end of the electrode body 100, and may also be disposed on a side of the electrode body 100 according to the mounting position of the electrode body 100. The surface of the convex portion 5 is specifically coated with a conductive coating for facilitate signal acquisition.

Specifically, the insulating anti-interference material layer 9 includes an insulating coating layer 400 as a top layer and a metal mesh layer 300 and/or a metal foil layer 200 disposed between the insulating coating layer 400 and the electrode body 100. In one embodiment, the insulating anti-interference material layer 9 includes an insulating coating layer 400 as a top layer and a metal mesh layer 300 disposed between the insulating coating layer 400 and the electrode body 100. In another embodiment, the insulating anti-interference material layer 9 includes an insulating coating layer 400 as a top layer and a metal foil layer 200 disposed between the insulating coating layer 400 and the electrode body 100. In another embodiment, the insulating anti-interference material layer 9 includes an insulating coating layer 400 as a top layer and a metal mesh layer 300 and a metal foil layer 200 disposed between the insulating coating layer 400 and the electrode body 100. For example, the metal mesh layer 300 and the metal foil layer 200 are stacked with each other, but the sequence of the metal mesh layer 300 and the metal foil layer 200 is not limited. For example, the metal foil layer 200, the metal mesh layer 300, and the insulating coating layer 400 are sequentially stacked from the surface of the electrode body 100, or the metal mesh layer 300, the metal foil layer 200, and the insulating coating layer 400 are sequentially stacked from the surface of the electrode body 100.

Since the electromagnetic interference (EMI) is mainly low-frequency interference, motors, fluorescent lamps, and power supply cords are common electromagnetic interference sources. Radio frequency interference (RFI) is high-frequency interference, mainly wireless frequency interference, including radio, television broadcasting, radar, and other wireless communications. For suppressing electromagnetic interference, the most effective method is using a woven layer (e.g., to shield electromagnetic interference by using the metal mesh layer 300), since it has a relatively low critical resistance. For suppressing radio frequency interference, the most effective shield method is using a metal foil (e.g., the metal foil layer 200), since the gaps generated by the metal mesh shield allow high-frequency signals to freely enter and exit. For high and low frequency mixed interferences, a shielding method by using a combined layer of the metal foil layer 200 and the metal mesh layer 300 may have an excellent shielding effect. Therefore, the arrangement of the insulating anti-interference material layer 9 may be selected according to the frequency ranges of to be shielded.

Specifically, the metal foil layer 200 is fixed to the surface of the electrode body 100, and the metal mesh layer 300 is fixed to the surface of the metal foil layer 200. That is, the metal foil layer 200, the metal mesh layer 300, and the insulating coating layer 400 are sequentially stacked from the surface of the electrode body 100. The specific fixed connection method between two adjacent layers may adopt the conventional fixed method in the art. For example, the metal foil layer 200 is welded to the electrode body 100, and the metal mesh layer 300 is welded to the metal foil layer 200.

Specifically, the metal mesh layer 300 is a copper mesh layer, and the metal foil layer 200 is a copper foil layer. Copper mesh and copper foil may have an excellent effect in suppressing electromagnetic interference and radio frequency interference, and have a low price and thus reduce the cost. According to the actual situation, the metal mesh layer 300 may also be a silver mesh, and the metal foil layer 200 may also be other shielding materials such as silver layer. The metal mesh layer 300 has a mesh number of 200 to 400, a wire diameter of 20 μm to 70 μm, and the material thereof is one of copper, bronze, and brass. The metal foil layer 200 has a thickness of 10 μm to 30 μm, the material thereof is one of copper, bronze, and brass.

The insulating coating layer 400 may specifically be a plastic layer. Plastic has an excellent insulating effect and is easy to be molded. Specifically, the metal foil layer 200, the metal mesh layer 300, and the electrode body 100 may be first welded and fixed into an integral piece, and then the insulating powder is charged by using a high-voltage electrostatic apparatus, and sprayed onto the surface of the integrated piece under the action of an electric field, and leveled and solidified to form the insulating coating layer 400. When plastic powder is sprayed onto the surface of an integrated piece, the powder may be uniformly adsorbed on the surface of the integrated piece to form a powdery coating layer, and then the powdery coating layer is leveled and solidified into one dense protective coating layer after high-temperature baking. The specific insulating coating layer 400 is an organic coating. The thickness of the insulating coating layer 400 is greater than the total thickness of the metal foil layer 200 and the metal mesh layer 300. The specific total thickness of the insulating coating layer 400, the metal foil layer 200, and the metal mesh layer 300 is in a range of 80 μm to 2000 μm. Specifically, the materials of the insulating coating layer 400 include Teflon, acrylic powder, and polyester powder. The high-temperature curing process preferably performed under a curing temperature of 170° C. to 200° C. during a curing time of 5 to 60 minutes.

In order to ensure a reliability of the connection between the insulating anti-interference material layer 9 and the electrode body 100, an adhesive layer 500 is further provided for bonding and fixing the electrode body 100 with the metal foil layer 200 or the metal mesh layer 300. That is, when the electrode body 100 is connected to the metal foil layer 200, the electrode body 100 and the metal foil layer 200 are fixed by the adhesive layer 500. When the electrode body 100 is connected to the metal mesh layer 300, the electrode body 100 and the metal mesh layer 300 are fixed by the adhesive layer 500. When the metal foil layer 200 or the metal mesh layer 300 is welded to the electrode body 100, the connection reliability may be improved by using the adhesive layer 500.

Specifically, the adhesive layer 500 is a plastic layer formed by spray molding. That is, a plastic layer is formed between the electrode body 100 and the metal foil layer 200 or the metal mesh layer 300 by spray molding, to combine the electrode body 100 and the metal foil layer 200 or the metal mesh layer 300. When the insulating coating layer 400 is a plastic layer, by spraying and coating plastic powder onto the surface of the electrode body 100 attached with the metal foil layer 200 and/or the metal mesh layer 300, and forming a plastic layer on the surface of the metal foil layer 200 or the metal mesh layer 300 and between the electrode body 100 and the metal foil layer 200 or the metal mesh layer 300 respectively, the plastic layer on the surface thereof may have an insulating function, while the plastic layer in contact with the electrode body 100 has a connecting function. The above structure is easy to process and has a reliable connection. According to actual situation, other conventional methods may also be used to combine the electrode body 100 and the metal foil layer 200 or the metal mesh layer 300.

On the basis of the above embodiments, the electrode body 100 serves as the button 2 of the wearable device, and the insulating anti-interference material layer 9 has a second notch at a position corresponding to a bottom end of the electrode body 100, and the electrode body 100 is exposed by the second notch to contact and electrically connect with the tactile switch 6 of the wearable device. That is, the button 2 is functioned as an electrode to acquire voltage signals. In order to ensure effective signal transmission by the button 2, a second notch is provided on the insulating anti-interference material layer 9. The second notch may be specifically positioned at a position of the insulating anti-interference material layer 9 corresponding to the bottom of the electrode body 100 to expose the electrode body 100, so that it is capable of contacting and conducting with the tactile switch 6. Specifically, when the electrode body 100 contacts the tactile switch 6, the voltage is measured and the signal is transmitted to the data processing unit of the device. When the biological monitoring electrode is used for electrocardiogram monitoring, upon the button 2 contacts the tactile switch 6, the voltage is measured, and the signal is transmitted to the data processing unit to obtain the required biological information, such as the user's electrocardiogram (ECG) in combination with the voltage measured by the electrode at the bottom of the device housing 1.

Based on the biological monitoring electrode provided in the above embodiments, the present disclosure further provides a wearable device, the wearable device includes any one or more of the biological monitoring electrodes in the above embodiments. Since the wearable device uses the biological monitoring electrode in the above embodiments, the wearable device may have the beneficial effects of the biological monitoring electrodes mentioned in the above description.

Specifically, the wearable device may be wrist mounted devices such as watches and bracelets, or may be other wearable devices such as head-worn display device.

Specifically, as shown in FIG. 1, the button 2 includes a sliding column, and the device housing 1 has a button support 3 for supporting the button 2. The button support 3 has a mounting groove that fits with the sliding column, and the sliding column slides along the mounting groove. A sealing ring 4 is provided between the sliding column and the mounting groove, and specifically, the sealing ring 4 for sealing is an O-ring and may be fluor rubber material. The device housing 1 is provided with a substrate 8 therein, and the button support 3 is fixedly mounted on the substrate 8. Specifically, the button support 3 may be fixed to the substrate 8 by using waterproof double-sided adhesive. The materials of key holder 3 may be stainless steel, ceramic, plastic, titanium and alloys thereof.

The various embodiments in the present specification are described in a progressive manner, and each embodiment focuses on the differences from other embodiments, and the same and similar parts between the various embodiments may be referred to each other.

The above description of the disclosed embodiments enables any person skilled in the art to implement or make use of the present disclosure. Various modifications to these embodiments will be readily apparent to those skilled in the art, and the generic principles defined herein may be implemented in other embodiments without departing from the spirit or scope of the present disclosure. Therefore, this application is not intended to be limited to the embodiments shown herein, but is to be accorded the widest scope consistent with the principles and novel features disclosed herein.

1. A biological monitoring electrode comprising an electrode body, wherein a surface of the electrode body is coated with an insulating anti-interference material layer, the insulating anti-interference material layer has a first notch, and the electrode body is exposed by the first notch to be contacted with a user's skin to acquire biological information.

2. The biological monitoring electrode according to claim 1, wherein the electrode body has a convex portion protruding from the first notch.

3. The biological monitoring electrode according to claim 1, wherein the insulating anti-interference material layer comprises an insulating coating layer as a top layer and a metal mesh layer and/or a metal foil layer disposed between the insulating coating layer and the electrode body.

4. The biological monitoring electrode according to claim 3, wherein the metal foil layer is fixed to a surface of the electrode body, and the metal mesh layer is fixed to a surface of the metal foil layer.

5. The biological monitoring electrode according to claim 3, wherein the metal mesh layer is a copper mesh layer, and the metal foil layer is a copper foil layer.

6. The biological monitoring electrode according to claim 3, wherein the insulating coating layer is a plastic layer.

7. The biological monitoring electrode according to claim 3, further comprising an adhesive layer for bonding and fixing the electrode body to the metal foil layer or the metal mesh layer.

8. The biological monitoring electrode according to claim 7, wherein the adhesive layer is a plastic layer formed by spray molding.

9. The biological monitoring electrode according to claim 1, wherein the electrode body serves as a button for a wearable device, and the insulating anti-interference material layer has a second notch at a position corresponding to a bottom end of the electrode body, the electrode body is exposed by the second notch to contact and electrically connect with a tactile switch of the wearable device.

10. A wearable device comprising the biological monitoring electrodes according to claim 1.
